(19) United States
(12) Patent Application Publication
(10) Pub. No.: US 2003/0180226 A1
(43) Pub. Date: Sep. 25, 2003

(54) Anti-bacterial sneeze spray

(76) Inventor: Pauline A. Haughton, Forestville, MD (US)

Correspondence Address: PAULINE ANGELA HAUGHTON, 1923 ADDISON ROAD SOUTH, FORESTVILLE, MD, 20747, US

(21) Appl. No.: 10/350,396

(22) Filed: Jan. 23, 2003

Related U.S. Application Data
(60) Provisional application No. 60/350,333, filed on Jan. 23, 2002.

(51) Int. Cl. A61L 9/04

(52) U.S. Cl. 424/45

(57) ABSTRACT

In an environment such as hospitals, plant nurseries, paper factories, daycare centers, public restrooms and places where the air you breath might be contaminated or suspect a preventative aide such as this product, will help in this area. Sneezing for a purpose!

### SPECIFICATIONS

I claim the benefit of an earlier U.S. Provisional application Serial No. 60/350,333, filing date Jan. 23, 2002, Title—Anti-Bacterial Sneeze Spray—Pauline A. Haughton

What I claim as my invention is a product that when sprayed into the air and breathed in kills off certain bacteria and germs; then causing a sneezing effect simultaneously, thus sneezing out germs, bacteria, spores and dust. Causing a clearing, cleaning, disinfecting effect.

### DETAILS

Infusion of Garlic—anti-viral, anti-bacterial, disinfectant—40%

Goldenseal Root, Echinacea, yarrow extract—Anti viral, anti bacterial, disinfectant 20%

Capsicum Extract—sneeze inducer, 10%

Cinnamon, Cloves Extract—odor control—20%

Jamaican Ginger Extract—Sneeze inducer—10%

Alcohol—Preservative—25%

Water total volume—75%

All ingredients are steeped in water, cooled, stained, then mixed together in the above percentage, with alcohol as preservative. Ready to spray.

### BACKGROUND OF INVENTION

Studies have shown that most viral infections i.e., colds, flu etc., starts within the sinus area. Bacteria, germs, air-borne spores, and pollutants can be harmful to health if left unprotected. This spray was developed to help as a defense for our sinuses and good health. The idea is sneezing for the purpose of getting rid of and clearing out unwanted foreign matter and pollutants from the sinus by killing them then sneezing, preventing harm to ones health.

1. What I claim as my invention is a product that when sprayed into the air and breathed in kills off certain bacteria and germs; then causing a sneezing effect simultaneously, thus sneezing out germs, bacteria, spores and dust. Causing a clearing, cleaning, disinfecting effect.
